(19) United States
(12) Patent Application Publication
(10) Pub. No.: US 2013/0095518 A1
(43) Pub. Date: Apr. 18, 2013

(54) METHOD FOR PRODUCING TUMOR CELLS FROM NORMAL MAMMARY EPITHELIAL CELLS

(71) Applicants: Tsuyoshi Akagi, Hyogo (JP); Taiko Sukezane, Hyogo (JP)

(72) Inventors: Tsuyoshi Akagi, Hyogo (JP); Taiko Sukezane, Hyogo (JP)

(73) Assignee: EISAI R&D MANAGEMENT CO., LTD., Tokyo (JP)

(21) Appl. No.: 13/805,217

(22) PCT Filed: Jul. 7, 2011
(86) PCT No.: PCT/JP2011/066029

§ 371 Date: Dec. 18, 2012

(30) Foreign Application Priority Data
Jul. 7, 2010 (JP) 2010-154744

(51) Int. Cl. C12N 5/09

(52) U.S. Cl. CPC C12N 5/0693
USPC 435/29; 435/467; 435/325; 800/10; 435/363; 435/352

(57) ABSTRACT

An object of the present invention is to provide a method for producing tumor cells from cells derived from normal cells without using hTERT.
The present invention provides a method for producing tumor cells by carrying out the following treatments (1) and (2) on normal mammary epithelial cells:
(1) elimination of p53 function; and
(2) introduction v-Src gene.

### TECHNICAL FIELD

The present invention relates to a method for producing tumor cells based on normal mammary epithelial cells without using TERT, tumor cells produced according to this method, and a method for screening antitumor agents using the tumor cells.

### BACKGROUND ART

Breast cancer is characterized as a tumor that occurs in mammary tissue, and 10% of women in Western countries are known to be afflicted with breast cancer at some point in their lives. In addition, approximately 20% of women with breast cancer end up dying due to the cancer. Moreover, although occurring at a lower rate (about 0.1%), breast cancer is also known to occur in men.

The mechanism of the occurrence of breast cancer is still not figured out systematically. Consequently, the current treatment of breast cancer is forced to rely on the surgical excision of cancerous tissue as a general rule, with chemotherapy and radiotherapy being limited to the roles of secondary treatment. Among the types of breast cancer, a type known as “triple negative” breast cancer, which is negative for expression of estrogen receptor, negative for expression of progesterone receptor and negative for erthythroblastic leukemia viral oncogene homolog 2 (ERBB2) protein, in particular advances rapidly and has a poor prognosis, and therefore an effective treatment method is needed.

In recent years, development of in vitro cancer model systems has been proceeding by genetically manipulating normal human cells. Although conventional cell lines established from tumor tissue contained unknown genetic abnormalities and therefore were not suitable for research on tumorigenesis induced by gene alterations, several in vitro cancer model systems as described above are useful for directly determining the effects of specific gene alterations on tumorigenesis. For example, Non-Patent Document 1 reports the first successful malignant transformation of mammary epithelial cells by introducing human telomere reverse transcriptase (hTERT) gene, SV40T antigen gene and HRAS^{V12} gene into normal mammary epithelial cells. In addition, Kendall, S. D. et al. succeeded in producing breast cancer cells by introducing hTERT gene, TP53 mutant gene, cyclin D1 gene, Cdk4 mutant gene and HRAS^{V12} gene into normal mammary epithelial cells (Non-Patent Document 2). Moreover, an example of malignant transformation of normal human mammary epithelial cells has been reported in which a different gene combination consisting of hTERT gene, TP53 mutant gene, HRAS^{V12} gene and PIK3CA mutant gene were introduced therein (Non-Patent Document 3).

However, despite the progress being made by these studies, there are currently hardly any reports describing the artificial production of breast cancer cells demonstrating a pathologically high degree of similarity with breast cancer cells isolated from patients. In particular, there have been no reports of successful cases of production of triple negative breast cancer cells.

Although cancer cell lines isolated from cancer patients are currently used in cancer cell research and the development anticancer agents, there are variations in the status of gene mutation between these cell lines since nearly all of such patients derived cancer cell lines have many unspecified genetic damages. Thus, when such cells are used in research on specific molecules or signal pathways, the experimental results obtained cannot be applied to different cell lines. In addition, since these cells have unspecified genetic damages, there are cases in which they cannot be used in experiments for evaluating the involvement of individual genes in malignant transformation.
Non-Patent Document 1: Elenbaas, B. et al., Genes Dev. 2001, 15: 50-65
Non-Patent Document 2: Kendall, S. Disean., Cancer Res. 2005, Nov. 1; 65(21): 9824-9828
Non-Patent Document 3: Zhao, J. J., Proc. Natl. Acad. Sci. USA 2006, 31; 103(44): 16296-16300

### DISCLOSURE OF THE INVENTION

With the foregoing in view, there is a need for the development of cancer model cells in which the status of gene mutation is defined, and particularly the development of cancer models cells for triple negative breast cancer.

The inventors of the present invention succeeded in inducing malignant transformation in normal human mammary epithelial cells by genetically manipulating those cells. Since the tumor cells obtained in this manner underwent malignant transformation as a result of genetic manipulation of normal cells, the status of gene mutation is defined. When these tumor cells obtained by the inventors of the present invention were observed in detail, they were revealed to exhibit histological characteristics of human breast cancer cells, and particularly those of triple negative breast cancer cells. The above discovery led to the present invention.

Namely, the present invention relates to that indicated below.

[1] A method for producing tumor cells by carrying out the following treatments (1) and (2) on normal mammary epithelial cells:

(1) elimination of p53 function; and

(2) introduction v-Src gene.

[2] The method described in [1], wherein forced expression of a cyclin-dependent kinase gene is further carried out.
[3] The method described in [2], wherein the cyclin-dependent kinase gene is Cdk4 gene.
[4] A method for producing tumor cells by carrying out the following treatments (1) to (3) on normal mammary epithelial cells:

(1) elimination of p53 function;

(2) introduction of EGFR mutant gene; and

(3) introduction of c-Myc gene.

[5] The method described in any one of [1] to [4], wherein the tumor cells are negative for expression of estrogen receptor, negative for expression of progesterone receptor, and negative for expression of ERBB2.
[6] The method described in any one of [1] to [5], wherein the normal mammary epithelial cells are mammalian, primate or rodent cells.
[7] Tumor cells produced according to the method described in any one of [1] to [6].
[8] A method for screening anti-tumor agents, this method comprising:

(a) contacting the tumor cells described in [7] with a candidate substance; and

(b) detecting growth inhibitory effects on the tumor cells.

[9] A cancer-bearing animal model transplanted with the tumor cells described in [7].
[10] A method for screening anti-tumor agents, this method comprising:

(a) contacting the cancer-bearing animal model described in [9] with a candidate substance; and

(b) detecting growth inhibitory effects on tumor cells.

Since tumor cells produced according to the method of the present invention undergo malignant transformation by genetically manipulating normal cells, the gene mutation status thereof is defined. Thus, according to the present invention, a model system can be provided that is extremely useful for evaluating the involvement of individual genes in the development of breast cancer. In addition, since tumor cells produced according to the method of the present invention demonstrate the phenotype of triple negative breast cancer, they have the potential to be extremely useful in the development of treatment methods and therapeutic drugs for triple negative breast cancer, which has conventionally been difficult to treat.

### BRIEF DESCRIPTION OF THE DRAWINGS

FIG. 1 is a drawing indicating the anatomical origin of normal human mammary epithelial cells in an embodiment of the present invention;

FIG. 2 is a drawing indicating a method for producing tumor cells from normal mammary epithelial cells according to an embodiment of the present invention;

FIG. 3 is a drawing indicating the results of confirming expression and function of genes introduced into tumor cells of the present invention by Western blotting, with FIG. 3A indicating the results for expression of Src gene, FIG. 3B indicating the results for expression of p53 gene, and FIG. 3C indicating the results for phosphorylation of tyrosine by introduced Src gene;

FIG. 4A is a graph indicating the tumor tissue formation ability of tumor cells of the present invention, while FIG. 4B depicts photographs showing tumor tissue formed by transplanting tumor cells of the present invention into nude mice;

FIG. 5 is a drawing indicating the pathological characteristics of tumor tissue formed by transplanting tumor cells of the present invention (HME/53/v-Src cells) into nude mice;

FIG. 6 is a drawing indicating the pathological characteristics of tumor tissue formed by transplanting tumor cells of the present invention (HME/53/Cdk4/v-Src cells) into nude mice;

FIG. 7 indicates the results of having formed a tumor mass by transplanting tumor cells of the present invention produced without introducing TERT gene into nude mice; and

FIG. 8 is a drawing indicating the pathological characteristics of tumor tissue formed by transplanting tumor cells of the present invention (HME/53/EGFR^{T790.L858R}/c-Myc cells) into nude mice.

### BEST MODE FOR CARRYING OUT THE INVENTION

The following provides a detailed explanation of the present invention. The following embodiments are intended to be exemplary for the purpose of explaining the present invention, and are not intended to limit the present invention thereto. The present invention can be carried out in various forms provided they do not deviate from the gist thereof.

Furthermore, all references, laid-open patent publications, examined patent publications and other patent documents cited in the present description are incorporated in the present description by reference. In addition, the present description includes contents described in the description and drawings of Japanese Patent Application No. 2010-154744 that serves as the basis for claiming priority of the present application and was filed on Jul. 7, 2010.

### 1. Method for Producing Tumor Cells

In a first aspect, the present invention provides a method for producing tumor cells by carrying out the following treatments (1) and (2) on normal mammary epithelial cells. Note that there are no particular limitations on the order in which treatment is carried out:

(1) elimination of p53 function; and

(2) introduction v-Src gene.

In the above-mentioned aspect, the method of the present invention may also include: forcibly expressing a cyclin-dependent kinase gene as a third treatment (3) in addition to the treatments described above.

In a second aspect thereof, the present invention provides a method for producing tumor cells by carrying out the following treatments (1) to (3) on normal mammary epithelial cells. Note that there are no particular limitations on the order in which treatment is carried out:

(1) elimination of p53 function;

(2) introduction of EGFR mutant gene; and

(3) introduction of c-Myc gene.

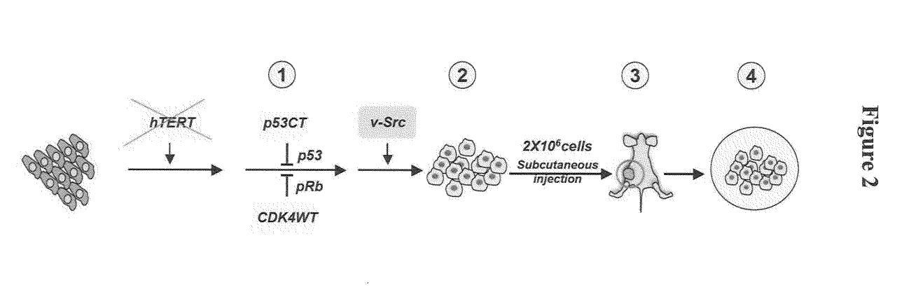

The method of the present invention is characterized by inducing malignant transformation of normal mammary epithelial cells without introducing TERT gene (see FIG. 2). There have previously been no reports of examples of successfully producing tumor cells without introducing TERT gene in a method for producing tumor cells from normal cells by expression of an exogenous gene.

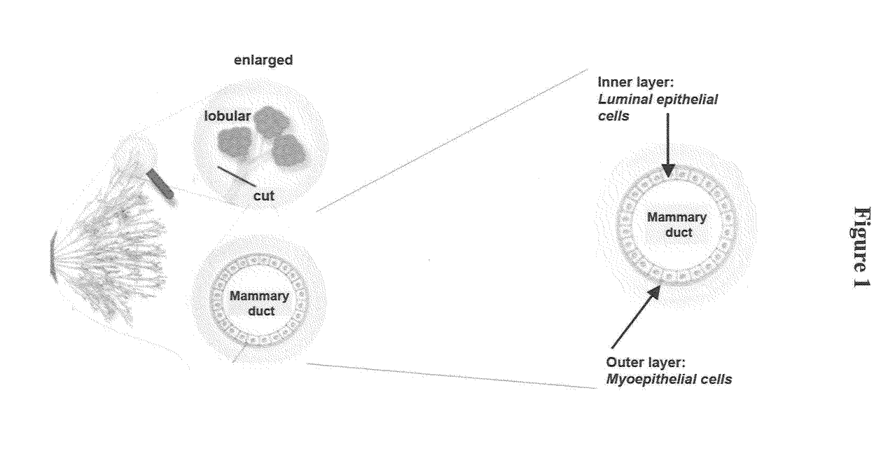

In the present invention, there are no particular limitations on the “normal mammary epithelial cells” provided they are normal cells derived from mammary epithelium. Mammary glands are known to be developed in mammals such as humans, monkeys and mice. In the present invention, “normal mammary epithelial cells” are preferably normal cells originating in mammalian mammary gland epithelium or mammary duct epithelium, more preferably normal cells originating in primate or rodent mammary gland epithelium or mammary duct epithelium, and even more preferably normal cells originating in human mammary duct epithelium or mammary duct epithelium. The normal cells are still more preferably normal cells originating in myoepithelium that forms the outer layer of the mammary duct (myoepithelial cells) (see FIG. 1). “Normal cells” refer to cells in a healthy state, or in other words, are in a state free of detectable diseases or abnormalities, while “normal human mammary epithelial cells” refers to human mammary epithelial cells in a state free of detectable diseases or abnormalities in the manner of human mammary epithelial cells from healthy subject, for example. In the following descriptions, “human mammary epithelial cells” are referred to as “human myoepithelial cells (HME cells)”.

HME cells harvested from human subjects may be used, or commercially available cells may be used (such as cells available from Lonza Inc. (Walkersville, Md., USA)).

“Elimination of p53 function” refers to p53 protein not having its inherent biological function.

p53 protein is encoded by TP53 gene, and TP53 gene is a gene that encodes p53 protein involved in activation of DNA repair protein, control of the cell cycle and induction of apoptosis, and functional abnormalities thereof are known to be related to the onset of various cancers (Lane, D. P. (1992) Nature 358: 15-16).

Various methods can be applied to eliminate p53 function, and although there are no particular limitations thereon provided it eliminates p53 function, examples of such methods include addition of p53 protein neutralizing antibody, cellular introduction of a gene that encodes that antibody, knockout of a gene that encodes p53 protein (to be referred to as “TP53 gene”), knockdown of TP53 gene by RNA interference (see Sato, M. et al. (Cancer Res. 66, (2006) 2116-2128)), and forced expression of dominant negative TP53 gene. Introduction of dominant negative TP53 gene (also referred to as “p53CT gene”) is preferably used in the present invention to eliminate p53 function. The nucleotide sequence of dominant negative TP53 gene can be ascertained by referring to the reference of Shaulian, E. et al. (Mol. Cell. Biol., December 1992; 12: 5581). The TP53 gene or p53 protein used when eliminating the function of p53 protein is preferably a gene or protein of mammalian origin, more preferably a gene or protein of primate origin, and even more preferably a gene or protein of human origin.

Furthermore, the function of other apoptosis-inducing proteins such as caspase-3, -8 to -10 or -12 may be eliminated instead of eliminating the function of p53. In this case as well, protein function can be eliminated using a similar method as that employed when eliminating the function of p53.

“v-Src gene” was discovered as a cancer-related gene originating in Rous sarcoma virus, which is a type of retrovirus, and the sequence thereof is described in Mayer, B. J. et al. (J. Virol. 1986 December; 60(3): 858-67).

“Cyclin-dependent kinase (Cdk) gene” is a gene that encodes protein involved in progression of the cell cycle, and the family of which it is a member is composed of Cdk1 to Cdk13. Although the Cdk gene introduced into normal mammary epithelial cells in the present invention may be a gene that encodes any of Cdk1 to Cdk13, a gene that encodes Cdk4 (Cdk4 gene) can be used preferably. Cdk4 is the binding partner of cyclin D1, and mutations of Cdk4 gene have been detected in various types of tumors (Zuo, L. et al. (1996), Nature Genet. 12, 97-99). The nucleotide sequence registered in the NCBI database is used for the nucleotide sequence of Cdk gene. In the present invention, Cdk gene is preferably a mammalian Cdk gene, more preferably a primate Cdk gene, and even more preferably a human Cdk gene.

“EGFR gene” is a gene that encodes epithelial growth factor receptor protein, and elevated expression of EGFR gene is observed in numerous cancers, including lung cancer, breast cancer, colorectal cancer, stomach cancer and brain cancer (Sharma, S. V. et al., Nature Rev. Cancer 2007; 7(3): 169-81).

In the present invention, “EGFR mutant gene” is an EGFR gene that has mutated from the wild type, and an example of which is a gene that encodes EGFR^{T790M.L858R} mutant protein (SEQ ID NO: 10) in which a threonine residue at position 790 has been substituted to methionine and a leucine residue at position 858 has been substituted to arginine. The nucleotide sequence of EGFR mutant gene can be produced according to the methods described in McCoy, M. S. et al. (Mol. Cell. Biol., (1984), 4, 1577-1582), Samuels, Y. et al. (Science (2004), 304, 554) or Scott, K. D. et al. (Cancer Res. (2007), 67, 5622-5627) based on the wild type nucleotide sequence registered in the NCBI database. In the present invention, EGFR gene is preferably mammalian EGFR gene, more preferably primate EGFR gene and even more preferably human EGFR gene.

“c-Myc gene” is a gene that encodes DNA binding factor protein involved in regulating the expression of various genes and DNA replication, or in other words, a transcription factor gene, abnormalities in the expression of c-Myc gene are suggested to be related to various cancers (Dominguez-Sola, D. et al. (2007) Nature 448, 445-451). The nucleotide sequence registered in the NCBI database is used for nucleotide sequence of c-Myc gene. In the present invention, c-Myc gene is preferably mammalian c-Myc gene, more preferably primate c-Myc gene and even more preferably human c-Myc gene.

The nucleotide sequences and peptide sequences of the various genes and proteins used in the examples to be subsequently described are indicated in the following Table 1, although not limited thereto.

TABLE 1

| | NCBI | | Sequence | Sequence |
|---|---|---|---|---|
| | Acces- | | No. | No. |
| Gene Name | sion No. | Species | (gene) | (protein) |
| TP53 gene | NM_000546.4 | Homo sapiens | — | — |
| Dominant | — | Homo sapiens | 1 | 2 |
| negative | | | | |
| TP53 gene | | | | |
| v-Src gene | — | Rous sarcoma | 3 | 4 |
| | | virus | | |
| Cdk4 gene | NM_000075.2 | Homo sapiens | 5 | 6 |
| EGFR gene | NM_005228.3 | Homo sapiens | — | — |
| EGFR mutant | — | Homo sapiens | 9 | 10 |
| gene | | | | |
| c-Myc gene | V00568.1 | Homo sapiens | — | — |

### Gene Introduction Method

In the case of introducing a gene into HME cells in the present invention, the gene is inserted into a suitable expression cassette in the form of an expression vector, and the HME cells are transformed with the expression vector. A suitable expression cassette at least contains the following constituents (i) to (iii):

(i) promoter capable of transcribing in HME cells;

(ii) gene ligated in-frame to the promoter; and

(iii) sequence encoding transcription termination and polyadenylation signal of RNA molecule.

Examples of promoters capable of transcribing in HME cells include, but are not limited to, CMV, CAG, LTR, EF-1α and SV40 promoters.

The above-mentioned expression vector may also have a selection marker expression cassette for selecting transformed HME cells in addition to the above-mentioned expression cassette. Examples of selection markers include, but are not limited to, positive selection markers such as neomycin resistance gene or hygromycin B phosphotransferase gene, expression reporters such as LacZ, green fluorescent protein (GFP) or luciferase gene, and negative selection markers such as herpes simplex virus thymidine kinase gene (HSV-TK) or diphtheria toxin A fragment (DTA).

Transformed HME cells can be easily selected with the above-mentioned markers. For example, in the case of cells introduced with a marker in the form of neomycin resistance gene, primary selection can be carried out by culturing in medium containing G418. In addition, in the case of containing a targeting vector in the form of a fluorescent protein gene such as GFP, in addition to selecting on the basis of drug resistance, sorting of cells expressing fluorescent protein may be carried out using a fluorescence-activated cell sorter (FACS).

Examples of expression vectors able to be used to introduce a gene into HME cells include expression vectors capable of introducing genes into cells, and commercially available expression vectors may also be used. Examples of such commercially available expression vectors include pEGFP-C1™ (Clontech), pCMV-HA™ (Clontech), pMSCVpuro™ (Clontech), pEF-DEST51™ (Invitrogen), pCEP4™ (Invitrogen) and ViraPower II Lentiviral Gateway System™ (Invitrogen). The expression vector can be introduced into HME cells by a known gene introduction method such as electroporation, microinjection, calcium phosphate method, lipofection or viral infection. Reference can be made to “Sambrook & Russell, Molecular Cloning: A Laboratory Manual, Vol. 3, Cold Spring Harbor Laboratory Press 2001”, for example, for details on gene introduction methods.

### 2. Tumor Cells

In the present invention, “tumor cells” refer to cells that autonomously undergo overgrowth in vivo, and may not only be tumor cells produced in the above-mentioned “method for producing tumor cells”, but may also be tumor cells isolated from a “cancer-bearing animal model” to be subsequently described or cells obtained by culturing, in vitro, tumor cells isolated from this cancer-bearing animal model.

Examples of tumor cells include cells contained in breast cancer tumors. In one embodiment of the present invention, a preferable form of the tumor cells is breast cancer cells. The tumor cells are more preferably triple negative breast cancer cells negative for all of the markers consisting of estrogen receptor (ER), progesterone receptor (PgR) and ERBB2 (also known as “HER2”) protein.

In the case of confirming malignant transformation of cells, the test cells are subcutaneously injected into a suitable animal model and confirming malignant transformation by observing the formation of a tumor mass.

A mammal other than a human is preferable for the animal model, while an immunosuppressed mammal is particularly preferable. Examples of immunosuppressed mammals include, but are not limited to, nude rats and nude mice.

Another example of a method for confirming malignant transformation of cells consists of culturing test cells on soft agar and observing their colony formation (soft agar colony formation assay method). As a specific example thereof, HME cells introduced with TP53 mutant gene and v-Src gene are disseminated in soft agar medium after adjusting to a constant cell concentration followed by observation of cell growth rate. Reference can be made to Tanaka, S. et al., Proc. Natl. Acad. Sci. USA, 94: 2356-2361, 1997 for details on the soft agar colony formation assay method.

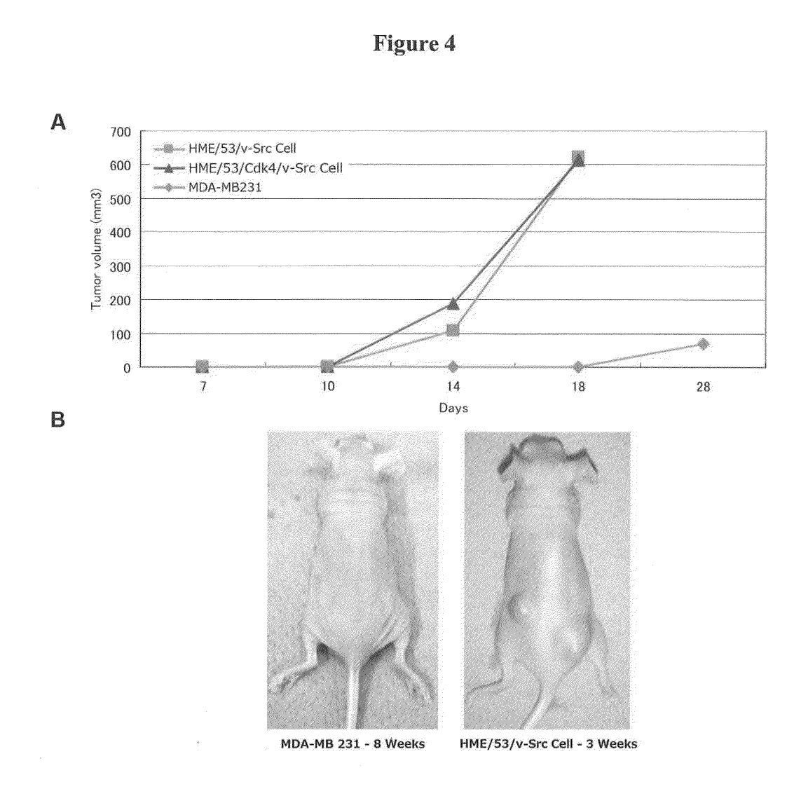

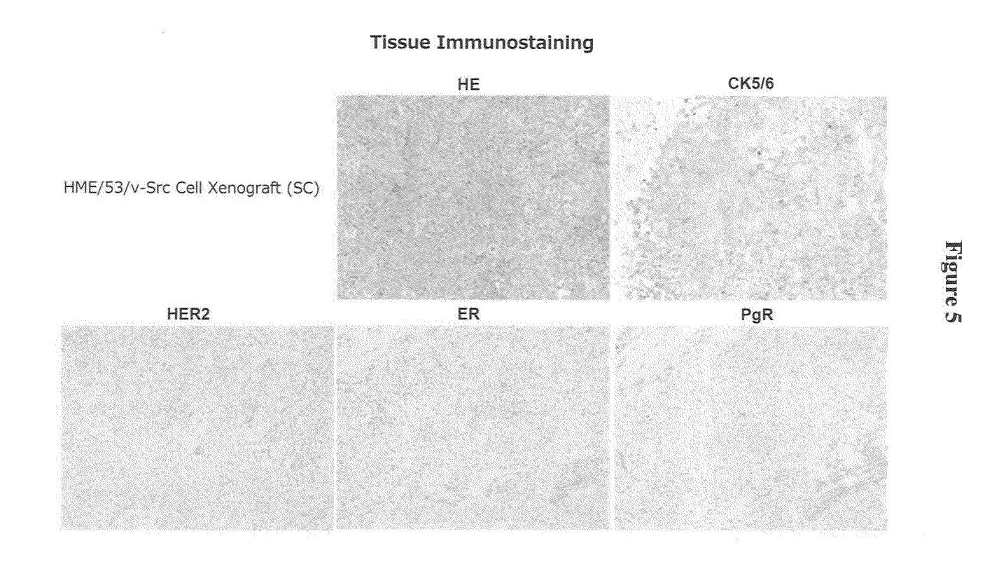

Tumor cells obtained in the present invention (to be referred to as “tumor cells of the present invention”) are characterized by having extremely high proliferation ability. As shown in FIG. 4A, tumor cells of the present invention have a remarkably high proliferation ability in comparison with human breast cancer cell line MDA-MB231 cells (ATCC HTB-26). In addition, in an embodiment thereof, tumor tissue obtained by transplanting the tumor cells of the present invention into an animal model were ER(−), PgR(−) and ERBB2(−), were ER(−), PgR(−) and HER2 (−) in the case of using human mammary epithelial cells, and exhibited the morphology of triple negative breast cancer cells (FIGS. 5, 6 and 8).

In tumor cells observed in human triple negative breast cancer, since the hormone receptors of ER and PgR are not expressed, and since the receptor tyrosine kinase of HER2 is also not expressed, hormone therapy or chemotherapy cannot be expected to be therapeutically effective, and triple negative breast cancer is considered to be the type of breast cancer that is most difficult to treat for this reason.

The tumor cells of the present invention have both a high tumorigenesis rate and high proliferation ability. In the case of forming a tumor by transplanting the tumor cells of the present invention into an animal model in particular, tumors are formed in the animal model that are pathologically extremely similar to tumor tissue spontaneously arising from the mammary gland (duct) epithelial cells in the animal species. For example, in the case of transplanting human mammary gland epithelial cells, tumors are formed in an animal model that are pathologically extremely similar to cancer tissue isolated from human cancer patients, and particularly triple negative breast cancer tissue. Thus, the tumor cells of the present invention have the potential to be extremely useful in the development of treatment methods or therapeutic drugs for triple negative breast cancer.

### 3. Cancer-Bearing Animal Model

In the present invention, a “cancer-bearing animal model” refers to an animal in which a tumor mass has been formed by transplanting the above-mentioned tumor cells into an animal model other than a human. A preferable example of an animal model is a mammal other than a human, examples of which include, but are not limited to, mice, rats, pigs, dogs, monkeys, hamsters and rabbits. Among these animal models, mammals are preferable, and primates or rodents are more preferable. Among these, immunosuppressed mammals are particularly preferable, while immunosuppressed primates or rodents are even more preferable. Although immunosuppressed mammals can be produced by administering an immunosuppressant such as cyclosporin to an ordinary mammal, mammals in which immunity has been congenitally suppressed based on genetic background are preferable. Examples of mammals in which immunity has been congenitally suppressed include, but are not limited to, nude rats and nude mice.

There are no particular limitations on the method used to transplant the tumor cells into the animal model. A method conventionally used corresponding to the animal model to be transplanted with the tumor cells may be suitably selected. Reference can be made to Genetic Induction of Tumorigenesis in Swine, Oncogene 26, 1038-1045 (Sep. 11, 2006), for example, for examples of transplanted animal models other than mice. From the viewpoint of ease of re-excision of the transplanted tumor cells, transplantation is preferably carried out by subcutaneous injection or intraperitoneal injection, while local transplantation is preferable from an anatomical viewpoint.

### 4. Method for Screening Antitumor Agents

The present invention provides a method for screening anti-tumor agents.

In a first aspect thereof, as the screening method of the present invention, a method for screening anti-tumor agents is provided that includes:

(a) contacting the tumor cells of the present invention with a candidate substance; and

(b) detecting growth inhibitory effects on the tumor cells.

In addition, in a second aspect thereof, as the screening method of the present invention, a method for screening anti-tumor agents is provided that includes:

(a) contacting a cancer-bearing animal model transplanted with the tumor cells of the present invention with a candidate substance; and

(b) detecting growth inhibitory effects on the tumor cells.

In the above-mentioned descriptions, “contacting the tumor cells with a candidate substance” or “contacting a cancer-bearing animal model transplanted with tumor cells with a candidate substance” refers to the candidate substance approaching the tumor cells to a degree that it interacts with molecules on the surface thereof or binds with those molecules, or adjusting conditions at which the candidate substance is taken into the tumor cells. In the case the tumor cells are tumor cells such as cultured cells, a candidate substance can be allowed to contact the cells by adding the candidate substance to a culture medium contacted by the cells at a fixed concentration or more. On the other hand, in the case the tumor cells have been transplanted into an animal body, namely in the case of a cancer-bearing animal model transplanted with the tumor cells, a candidate substance can be allowed to contact the tumor cells by administering the candidate substance to the animal at a fixed dosage. In this case, although there are no particular limitations on the administration route provided it is a route that is commonly employed to administer a candidate substance, and specific examples include oral, sublingual, pernasal, intrapulmonary, alimentary, percutaneous, instillation, intravenous injection, subcutaneous injection, intramuscular injection, intraperitoneal injection, local injection and surgical transplant, with oral administration, intraperitoneal injection and intravenous injection being preferable.

Examples of candidate substances include compounds drugs for which antitumor effects have already been confirmed as well as compounds, polypeptides, nucleic acids, antibodies and low molecular-compounds having the potential to exhibit antitumor effect. Specific examples of such candidate substances include, but are not limited to, metabolic antagonists (such as 5-fluorouracil (5-FU)), folate metabolism antagonists (including dihydropteroic acid synthase inhibitors such as sulfadiazine or sulfamethoxazole and dihydrofolate reductase inhibitors (DHFR inhibitors) such as methotrexate, trimethoprim or pyrimethamine), pyrimidine metabolism inhibitors (including thymidylate synthase inhibitors such as 5-FU or flucytosine (5-FC)), purine metabolism inhibitors (including IMPDH inhibitors such as 6-mercaptopurine and the prodrugs such as azathioprine), adenosine deaminase (ADA) inhibitors (such as pentostatin), ribonucleotide reductase inhibitors (such as the ribonucleotide reductase inhibitor, hydroxyurea), nucleotide analogs (including purine analogs such as thioguanine, fludarabine phosphate or cladribine as well as pyrimidine analogs such as cytarabine or gemcitabine), L-asparaginase, alkylating agents (including nitrogen mustard agents such as cyclophosphamid, melphalan or thiotepa, platinating agent preparations such as cisplatin, carboplatin or oxaliplatin, and nitrosourea drugs such as dacarbazine, procarbazine or ranimustine), antitumor antibiotics (such as sarcomycin, mitomycin C, doxorubicin, epirubicin, daunorubicin or bleomycin), topoisomerase inhibitors (such as irinotecan, nogitecan, doxorubicin, etoposide, levofloxacin or ciprofloxacin), microtubule polymerization inhibitors (such as vinblastine, vincristine or vindesine), colchicine, microtubule depolymerization inhibitors (such as paclitaxel or docetaxel), molecular-targeted agents (such as trastuzumab, rituximab, imatinib, gefitinib, bortezomib or erlotinib), steroids such as dexamethasone, finasteride, aromatase inhibitors, tamoxifen and combinations thereof.

Growth inhibitory effects on tumor cells can be confirmed by producing two systems of cultures of the same cells disseminated in culture dishes at the same cell density and cultured under the same conditions or two cancer-bearing animal models of the same strain transplanted with the same number of cells at the same cell density, contacting the above-mentioned candidate substance with cells of one of the systems (sample), not contacting the candidate substance with cells of the other system (control), observing proliferation of the tumor cells of both systems, and measuring and comparing the number of tumor cells contained in each of the two systems after a fixed period of time has elapsed.

In the case of screening the tumor cells of the present invention in the form of tumor cells such as cultured cells, tumor growth effects can be confirmed by contacting a candidate substance with the above-mentioned sample cells, measuring the number of sample cells and the number of control cells with a cell counter and the like, and comparing the numbers of each of the cells.

In the case of screening the tumor cells of the present in the form of transplanting into an animal model, namely in the case of screening in the form of an animal model in which the tumor cells have been transplanted, tumor growth effects can be confirmed by administering a candidate substance to the animal model of a sample system, extracting tumor tissue from animals of the sample system and control system, and measuring and comparing the number of cells contained in the tumor tissue from the sample system and control system. Alternatively, tumor growth effects can also be confirmed by extracting the above-mentioned tumor tissue, and comparing the volume of the tumor tissue between a sample system and a control system. Tumor volume can be determined with the following equation.

Tumor volume=ab²/2

(a: horizontal width, b: length)

Alternatively, tumor growth effects can also be confirmed by administering a candidate substance to animals of a sample system and a control system, and comparing rate at which a measurable tumor mass is formed at the transplanted location of the tumor cells between the sample system and the control system.

The animal model is the same as the animal model described in section 3 entitled “Cancer-Bearing Animal Model”. Although there are no limitations on the method used to transplant tumor cells into the animal model, subcutaneous injection or intraperitoneal injection is preferable in consideration of the ease of re-excision of the transplanted tumor cells.

In the case the rate of increase of tumor cells of the sample system is less than the rate of increase of tumor cells of the control system, the candidate substance used can be judged to have antitumor effects. Alternatively, in the case multiple sets of data on the rate of increase under fixed conditions are already available for the tumor cells of the present invention, an assessment may also be made by statistically processing that data, and comparing standard values derived from the resulting average values, standard deviations and the like.

Although average values or standard deviations and the like of tumor growth rates can be obtained by various statistical methods, more specifically, these values can be determined by two-way ANOVA using statistical processing software such as IBM SPSS Statistics 18 (SSPS) using the initial number of cells and cell density at the time of cell dissemination as parameters in the case of cultured cells, or using body weight of an animal model at the time of transplant and the number of transplanted tumor cells in the case of transplanted cells. Analysis accuracy can be further improved by using the growth rate of tumor cells obtained by carrying out the method of the present invention as new data and adding to the population used for statistical analysis to increase statistical parameters.

Since the tumor cells of the present invention demonstrate characteristics that are pathologically extremely similar to breast cancer cells isolated from actual patients, antitumor agents that have been confirmed to demonstrate growth inhibitory effects on tumor cells as determined with the screening method of the present invention are expected to demonstrate antitumor effects in cases of actually using in the treatment of cancer patients, and particularly breast cancer patients (such as triple negative breast cancer patients).

Although the following provides a detailed explanation of the present invention using an example thereof, the present invention is not limited to the aspects described in the example.

### Example

In the present example, tumor cells were produced by introducing the gene combinations shown in Table 1 to normal HME cells.

In the following descriptions, each of the produced tumor cells is indicated using the nomenclature shown in the following table corresponding to the type of gene introduced into the HME cells.

TABLE 2

| Genes Introduced into HME Cells | Tumor Cell Nomenclature |
|---|---|
| Dominant negative TP53 | HME/53 cells |
| (p53CT) gene | |
| Dominant negative TP53 gene | HME/53/v-Src cells |
| and v-Src gene | |
| Dominant negative TP53 gene | HME/53/Cdk4 cells |
| and Cdk4 gene | |
| Dominant negative TP53 gene, | HME/53/Cdk4/v-Src cells |
| Cdk4 gene and v-Src gene | |
| Dominant negative TP53 gene | HME/53/c-Myc cells |
| and c-Myc gene | |
| Dominant negative TP53 gene | HME/53/EGFR^{T790.L858R} cells |
| and EGFR^{T790.L858R} gene | |
| Dominant negative TP53 gene, | HME/53/EGFR^{T790.L858R}/c-Myc cells |
| EGFR^{T790.L858R} gene and | |
| c-Myc gene | |

The following provides a description of the experimental procedure carried out in the present example.

### Cell Culture

Normal human mammary epithelial cells derived from a 30-year-old Caucasian woman were purchased from Lonza Inc. (Walkersville, Md., USA)) for use as HME cells. The cells were cultured in a collagen-coated dish in serum-free MEGM medium (MEGM Bullet Kit, Lonza Inc.) containing various growth factors provided by Lonza Inc. The cells were cultured in a humid incubator maintained at 37° C. in a low oxygen environment (3% O₂ and 5% CO₂), and were used in the present example as HME cells.

Production of Retrovirus Vector and Introduction of Retrovirus Vector into HME Cells

Vectors for retrovirus expression were produced by incorporating the v-Src, Cdk4, dominant negative TP53, EGFR^{T790.L858R} and c-Myc genes shown in Table 1 into each of the vectors shown in the following Table.

TABLE 3

| Insert Gene | Expression Vector |
|---|---|
| v-Src | pCX4pur vector |
| | (GenBank Accession No: AB086386) |
| Cdk4 | pCX4bsr vector |
| | (GenBank Accession No: AB086384) |
| Dominant negative TP53 | pCX4.1hisD vector |
| | (GenBank Accession No: AB086389) |
| EGFR^{T790.L858R} | pCX4pur vector |
| | (GenBank Accession No: AB086386) |
| c-Myc | pCX4bleo vector |
| | (GenBank Accession No: AB086388) |

Virus vectors were produced by introducing the above-mentioned retrovirus expression vectors along with pGP and pE-eco plasmids purchased from Takara Bio Inc. (Shiga, Japan) into 293T cells. Subsequently, the above-mentioned virus vectors were infected into HME cells that expressed Ecotropic receptor (Eco VR).

The cells infected with the virus vectors were subjected to selection by culturing for 2 weeks in the presence of puromycin and bleomycin. The cultured cells were selected from groups of cultured cells exhibiting polyclonal proliferation when carrying out selection using either of the drugs.

Furthermore, HME cells expressing ecotropic receptor (Eco VR) were produced according to the procedure indicated below.

cDNA containing the entire encoding region of mouse ecotropic retrovirus receptor (S1c7a1, NM_{—}007513) was cloned by RT-PCR using the primers indicated below.

| (SEQ ID NO: 7) | |
|---|---|
| FW primer: | 5′-GATCCTCCCCAGTGAGAAGT-3′ |
| (SEQ ID NO: 8) | |
| RV primer: | 5′-CTCACTAGCCATCTGGAGTG-3′ |

The amplification product of the above-mentioned RT-PCR was incorporated in pCx4hyg vector (GenBank Accession No: AB086387). A virus vector was then produced by introducing this vector along with plasmids pGP and pE-Ampho purchased from Takara Bio Inc. (Shiga, Japan) into 293T cells, and the virus vector was infected into HME cells followed by carrying out selection by culturing for 2 weeks in the presence of hygromycin.

### Immunoblotting

Protein assay, SDS-PAGE and immunoblotting were carried out in accordance with the description of a previous reference (Akagi, T. et al. (2002), Mol. Cell. Biol., 22, 7015-7023). The signals of proteins exhibiting a positive immune response were visualized by chemiluminescence using the SuperSignal WestFemto reagent (Pierce Inc.). Anti-Src antibody, anti-phospho-Src (Tyr416) antibody and anti-p53 antibody were purchased from Cell Signaling Technology Inc., while anti-phospho-tyrosine antibody (4G10) was purchased from Millipore Corp.

### Xenograft Growth Experiment

A xenograft growth experiment was carried out using mice. More specifically, cell suspensions (single-cell suspensions) containing 1×10⁶ HME cells expressing the gene combinations shown in Table 1 were suspended in 50% Matrigel™ (BD Biosciences Inc., San Jose, Calif., USA) and subcutaneously injected into the flanks of 6-week- or 7-week-old female athymic nude mice (BALB/c nu/nu, Japan SLC Inc., Hamamatsu, Japan) or NOD-SCID mice. Following transplantation, the formation of a tumor mass was confirmed visually. During this experiment, formation of a tumor mass was judged to be negative when a volume of a tumor mass does not reach to a degree that it can be pinched between the fingers at 12 weeks after transplantation. Then, the dimensions of the tumors were measured after transplantation using a caliper, and tumor volume was calculated based on the following equation to estimate tumorigenicity.

Tumor volume=ab²/2

(a: horizontal width, b: length)

In addition, the rates at which a measurable tumor mass is formed at the transplanted location of HME cells transduced with each of the cancer-related genes were calculated as a tumorigenesis rate and used to estimate tumorigenicity.

### Histological Analysis and Immunohistochemical Staining

Xenografts transplanted in the above-mentioned xenograft growth experiment were removed on day 20 after transplantation. The xenografts were fixed in formalin and embedded in paraffin followed by slicing into sections and subjecting to hematoxylin and eosin (H&E) staining in accordance with the normal protocol. Immunohistochemical staining was then carried out using the following antibodies in accordance with a previous reference (Sasai, K. et al. (2008), Am. J. Surg. Pathol. 32, 1220-1227): cytokeratin 5/6 (CK5/6, Dako Corp., M7237), estrogen receptor (ER, Dako Corp., M7040), progesterone receptor (PgR, Dako Corp., M3569) and HER2 (Dako Corp., K5204).

More specifically, immunohistochemical staining was carried out according to the following procedure. Tissue sections having a thickness of 4 μm were deparaffinized with xylene and then dehydrated with ethanol. Antigen was retrieved by heating for 2 minutes in 10 mM citrate buffer (pH 6.0) in an autoclave. Endogenous peroxidase was deactivated by rehydrating the tissue sections with phosphate-buffered physiological saline containing 0.01% Tween 20 (PBST), and incubating with 0.3% hydrogen peroxide. After incubating the tissue sections overnight at 4° C. with primary antibody at a suitably diluted concentration and washing with PBST, the tissue sections were incubated for 30 minutes at room temperature in Envision Dual Link Solution (Dako Corp., Glostrup, Denmark). Next, the sections were treated with diaminobenzene (Dako Corp.) to visualize antigen-antibody reaction sites and subjected to nuclear staining by treating with hematoxylin for 90 seconds. Slides of the tissue sections were observed by sealing the tissue sections with a cover glass after mounting with Entellan Neu Reagent (Merck & Co., Whitehouse Station, N.J., USA).

### Experiment Results

[1] Confirmation of Expression of Genes Introduced into HME Cells

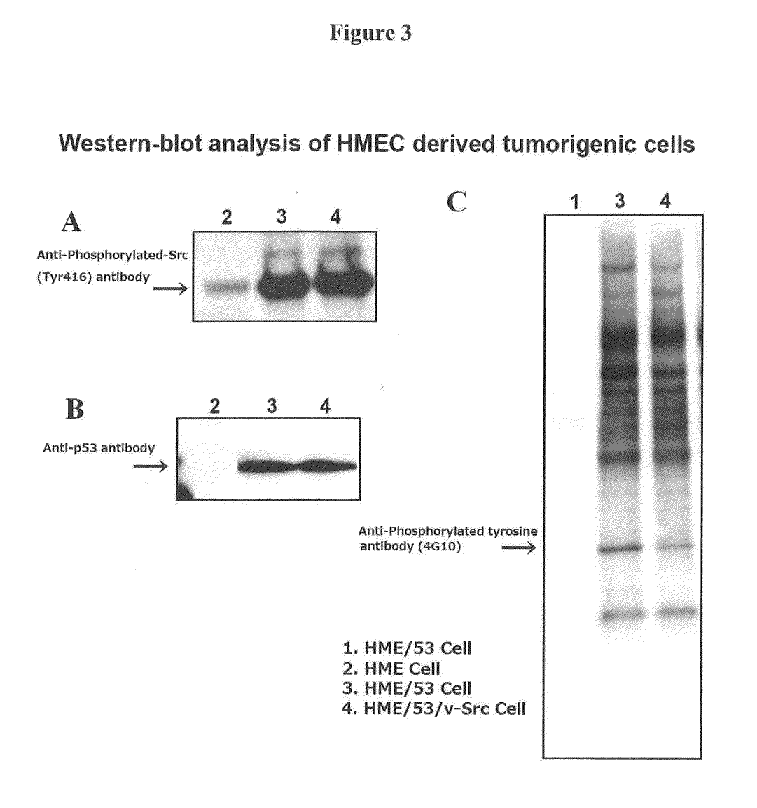

Expression of genes introduced into the HME cells was confirmed by immunoblotting. Expression of dominant negative TP53 gene and v-Src gene introduced into HME/453/v-Src cells and HME/53/v-Src cells was confirmed by antigen-antibody reaction using anti-phospho-Src antibody and anti-p53 antibody (respectively shown in FIGS. 3A and 3B). In addition, increased phosphorylation of tyrosine due to the function of v-Src gene was confirmed with anti-phospho-tyrosine antibody, thereby confirming that the target genes had been introduced and expressed (FIG. 3C).

### [2] Confirmation of Tumorigenicity of Produced Tumor Cells

The produced tumor cells were subcutaneously transplanted into nude mice to confirm tumorigenicity of the transplanted tumor graft (FIGS. 4, 7 and 8).

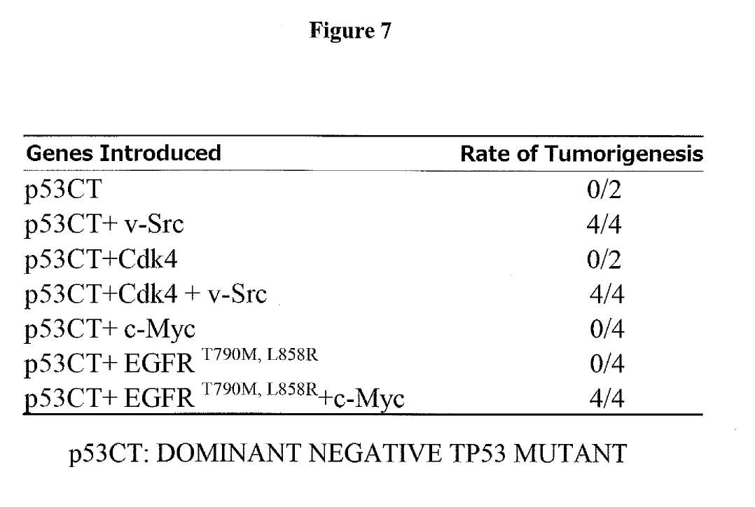

As shown in FIG. 7, formation of a tumor mass was not confirmed in those cells obtained by introducing dominant negative TP53 gene into HMEC cells (HME/53 cells) or in those cells obtained by introducing dominant negative TP53 gene and Cdk4 gene into HMEC cells (HME/53/Cdk4 cells).

As shown in FIGS. 4 and 7, the formation of a tumor mass of a size able to be pinched between the fingers was confirmed in those cells obtained by introducing dominant negative TP53 gene and v-Src gene into HMEC cells (HME/53/Cdk4/v-Src cells and HME/53/v-Src cells), and these cells demonstrated a tumorigenesis rate of 100%. In addition, as shown in FIGS. 4A and 4B, HME/53/Cdk4/v-Src cells and HME/53/v-Src cells clearly demonstrated a high level of proliferation ability since larger tumors were formed in a short period of time as compared with the case of conventionally used human breast cancer cell line MDA-MB231 cells.

As shown in FIG. 7, a tumor mass was not formed in the case of HME/53/EGFR^{T790.L858R} cells and HME/53/c-Myc cells obtained by respectively and independently introducing EGFR^{T790.L858R} gene or c-Myc gene into HMEC cells transduced with dominant negative TP53 gene (HME/53 cells).

On the other hand, formation of a tumor mass was confirmed in the case of HME/53/EGFR^{T790.L858R}/c-Myc cells obtained by introducing the combination of dominant negative TP53 gene, EGFR^{T790.L858R} gene and c-Myc gene, and these cells demonstrated a tumorigenesis rate of 100%.

### [3] Confirmation of Phenotype of Produced Tumor Cells

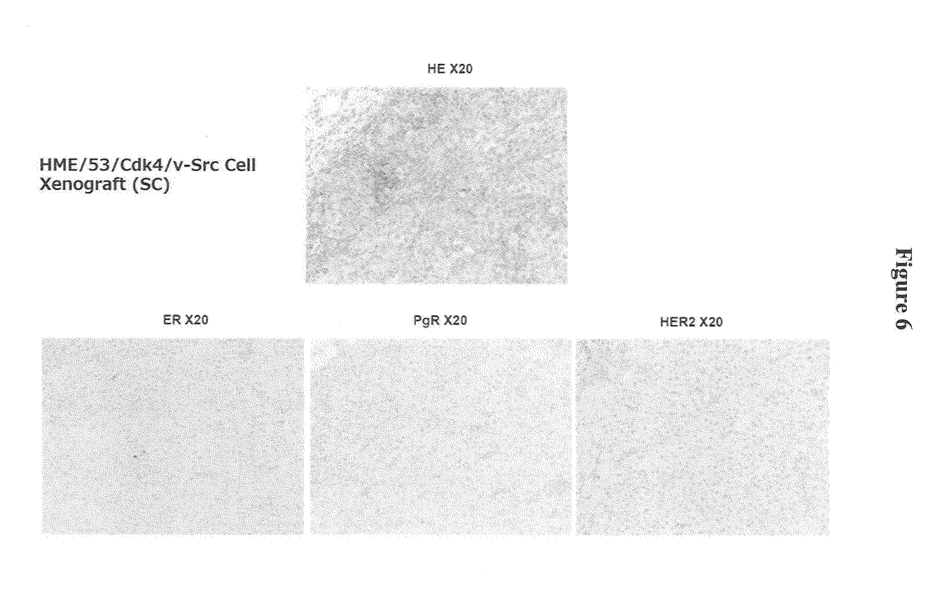

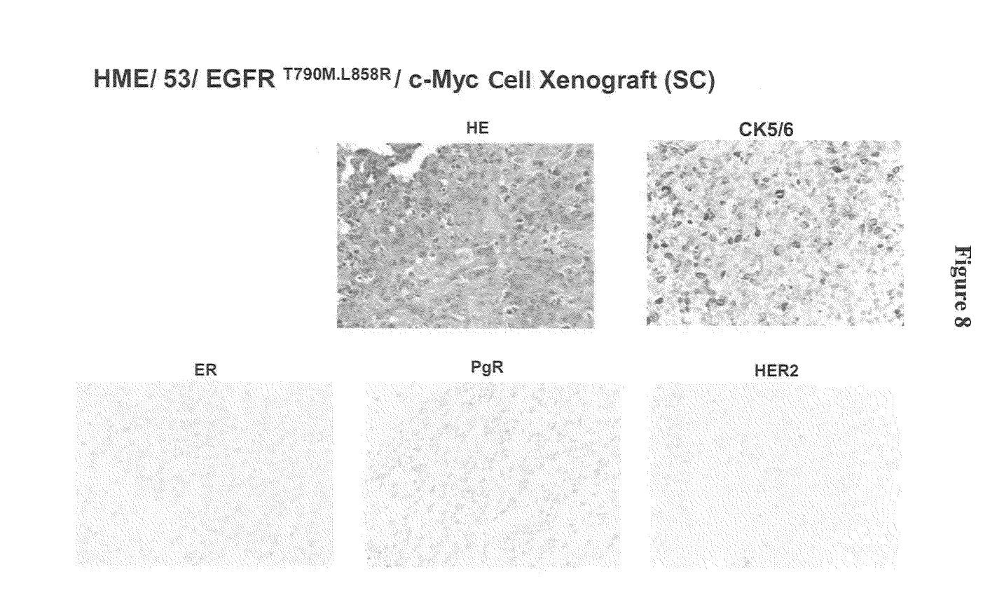

In order to confirm the phenotype of tumor tissue obtained from the xenograft growth experiment, the tumor tissue was immunostained with various cancer markers and observed. When a tissue section of a tumor formed by subcutaneous transplantation of HME/53/v-Src cells was stained with markers used for diagnosis in the clinical setting, the tissue was found to be ER(−), as shown in FIG. 5 PgR(−) and HER2(−) and positive for the Basal type breast cancer marker CK5/6 (CK5/6(+)), thereby demonstrating that HME/53/v-Src cells form tumors that are pathologically extremely similar to triple negative and Basal type breast cancer, which constitute the types of breast cancer that are the most difficult to treat. In addition, as shown in FIG. 6, tissue sections of tumors formed by subcutaneous transplantation of HME/53/Cdk4/v-Src cells were also ER(−), PgR(−) and HER2(−), thereby indicating that these cells similarly form tumors that are pathologically extremely similar to triple negative breast cancer. In addition, as shown in FIG. 8, tissue sections of tumors formed by subcutaneous transplantation of HME/53EGFR^{T790.L858R}/c-Myc cells were also ER(−), PgR(−) and HER2(−) and CK5/6(+) for the Basal phenotype breast cancer marker CK5/6, HME/53/EGFR^{T790.L858R}/c-Myc cells were demonstrated to form tumors that are pathologically extremely similar to triple negative and Basal type breast cancer, which constitute the types of breast cancer that are the most difficult to treat.

As indicated by the above-mentioned results, by introducing a combination of TP53 mutant gene and v-Src gene, a combination of TP53 mutant gene, v-Src gene and Cdk4 gene, or a combination of TP53 mutant gene, EGFR2 mutant gene and c-Myc gene into normal mammary epithelial cells, tumor cells can be produced without using hTERT gene that has been conventionally required to produce tumor cells. In addition, the present invention also enables the preparation of a human breast cancer model, and particularly a triple negative breast cancer model, that are extremely similar to breast cancer tissue isolated from human breast cancer patients.

### INDUSTRIAL APPLICABILITY

According to the present invention, tumor cells can be provided that exhibit pathological characteristics similar to those of human breast cancer cells. These tumor cells have the potential to be useful in research on the biochemical mechanism of breast cancer, identification of target molecules of breast cancer treatment, and screening and testing of antitumor agents.

Sequence Listing Free Text

SEQ ID NO: 7: Synthetic DNA

SEQ ID NO: 8: Synthetic DNA

Sequence Listing

1. A method for producing tumor cells by carrying out the following treatments (1) and (2) on normal mammary epithelial cells:
(1) elimination of p53 function; and
(2) introduction of a v-Src gene.

2. The method according to claim 1, wherein forced expression of a cyclin-dependent kinase gene is further carried out.

3. The method according to claim 2, wherein the cyclin-dependent kinase gene is a Cdk4 gene.

4. A method for producing tumor cells by carrying out the following treatments (1) to (3) on normal mammary epithelial cells:
(1) elimination of p53 function;
(2) introduction of an EGFR mutant gene; and
(3) introduction of a c-Myc gene.

5. The method according to claim 1, wherein the tumor cells are negative for expression of estrogen receptor, negative for expression of progesterone receptor and negative for expression of ERBB2.

6. The method according to claim 1, wherein the normal mammary epithelial cells are mammalian, primate or rodent cells.

7. Tumor cells produced by the method according to claim 1.

8. A method of screening anti-tumor agents
the method comprising:
(a) contacting the tumor cells according to claim 7 with a candidate substance; and
(b) detecting growth inhibitory effects on the tumor cells.

9. A cancer-bearing animal model transplanted with the tumor cells according to claim 7.

10. A method of screening anti-tumor agents,
the method comprising:
(a) contacting the cancer-bearing animal model according to claim 9 with a candidate substance, and
(b) detecting growth inhibitor effects on tumor cells.

11. The method according to claim 4, wherein the tumor cells are negative for expression of estrogen receptor, negative for expression of progesterone receptor, and negative for expression of ERBB2.

12. The method according to claim 4, wherein the normal mammary epithelial cells are mammalian, primate or rodent cells.

13. Tumor cells produced by the method according to claim 4.

14. A method of screening anti-tumor agents,
the method comprising:
(a) contacting the tumor cells according to claim 13 with a candidate substance; and
(b) detecting growth inhibitory effects on the tumor cells.

15. A cancer-bearing animal model transplanted with the tumor cells according to claim 13.

16. A method of screening anti-tumor agents,
the method comprising:
(a) contacting the cancer-bearing animal model according to claim 15 with a candidate substance, and
(b) detecting growth inhibitor effects on tumor cells.


SEQUENCE LISTING (10 sequences)

SEQ ID NO: 1 - DNA, 312 bp, Homo sapiens
  CDS (1)..(312)
atgtacccatacgatgttccagattacgctccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 2 - protein, 103 aa, Homo sapiens
MYPYDVPDYAPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 3 - DNA, 1581 bp, Rous sarcoma virus
  CDS (1)..(1581)
atggggagtagcaagagcaagcctaaggaccccagccagcgccggcgcagcctggagccacccgacagcacccaccacgggggattcccagcctcgcagacccccaacaagacagcagcccccgacacgcaccgcacccccagccgctccttcgggaccgtggccaccgagcccaagctcttcgggggcttcaacacttctgacaccgttacgtcgccgcagcgtgccggggcactggctggcggcgtcaccactttcgtggctctctacgactacgagtcctggattgaaacggacttgtccttcaagaaaggagaacggctgcagattgtcaacaacacggaaggtaactggtggctggctcattccctcactacaggacagacgggctacatccccagtaactatgtcgcgccctcagactccatccaggctgaagagtggtactttgggaagatcactcgtcgggagtccgagcggctgctgctcaaccccgaaaacccccggggaaccttcttggtccgggagagcgagacgacaaaaggtgcctattgcctctccgtttctgactttgacaacgccaaggggctcaatgtgaagcactacaagatccgcaagctggacagcggcggcttctacatcacctcacgcacacagttcagcagcctgcagcagctggtggcctactactccaaacatgctgatggcttgtgccaccgcctgaccaacgtctgccccacgtccaagccccagacccagggactcgccaaggacgcgtgggaaatcccccgggagtcgctgcggctggaggtgaagctggggcagggctgctttggagaggtctggatggggacctggaacggcaccaccagagtggccataaagactctgaagcccggcaccatgtccccggaggccttcctgcaggaagcccaagtgatgaagaagctccggcatgagaagctggttcaactgtacgcagtggtgtcggaagagcccatctacatcgtcattgagtacatgagcaaggggagcctcctggatttcctgaagggagagatgggcaagtacctgcggctgccacagctcgttgatatggctgctcagattgcatccggcatggcctatgtggagaggatgaactacgtgcaccgagacctgcgggcggccaacatcctggtgggggagaacctggtgtgcaaggtggctgactttgggctggcacgcctcatcgaggacaacgagtacacagcacggcaaggtgccaagttccccatcaagtggacagcccccgaggcagccctctatggccggttcaccatcaagtcggatgtctggtccttcggcatcctgctgactgagctgaccaccaagggccgggtgccatacccagggatgggcaacggggaggtgctggaccgggtggagaggggctaccgcatgccctgcccgcccgagtgccccgagtcgctgcatgaccttatgtgccagtgctggcggagggaccctgaggagcggcccactttcgagtacctgcaggcccagctgctccctgcttgtgtgttggaggtcgctgagtag

SEQ ID NO: 4 - protein, 526 aa, Rous sarcoma virus
MGSSKSKPKDPSQRRRSLEPPDSTHHGGFPASQTPNKTAAPDTHRTPSRSFGTVATEPKLFGGFNTSDTVTSPQRAGALAGGVTTFVALYDYESWIETDLSFKKGERLQIVNNTEGNWWLAHSLTTGQTGYIPSNYVAPSDSIQAEEWYFGKITRRESERLLLNPENPRGTFLVRESETTKGAYCLSVSDFDNAKGLNVKHYKIRKLDSGGFYITSRTQFSSLQQLVAYYSKHADGLCHRLTNVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVIEYMSKGSLLDFLKGEMGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMGNGEVLDRVERGYRMPCPPECPESLHDLMCQCWRRDPEERPTFEYLQAQLLPACVLEVAE

SEQ ID NO: 5 - DNA, 912 bp, Homo sapiens
  CDS (1)..(912)
atggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtga

SEQ ID NO: 6 - protein, 303 aa, Homo sapiens
MATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPE

SEQ ID NO: 7 - DNA, 20 bp, Artificial
  Synthetic DNA
gatcctccccagtgagaagt

SEQ ID NO: 8 - DNA, 21 bp, Artificial
  Synthetic DNA
ctcactagccatctggagtag

SEQ ID NO: 9 - DNA, 3633 bp, Homo sapiens
  CDS (1)..(3633)
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcatgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggcgggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 10 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLIMQLMPFGCLLDYVREHKDNIGSQYLLNWCVQIAKGMNYLEDRRLVHRDLAARNVLVKTPQHVKITDFGRAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA